(19) United States
(12) Patent Application Publication
(10) Pub. No.: US 2026/0114770 A1
(43) Pub. Date: Apr. 30, 2026

(54) SYSTEM AND METHOD FOR MULTIMODAL BIOMETRIC HUMOR SYNCHRONIZATION AND INTERPERSONAL VECTOR CORRELATION

(71) Applicant: Vikram Joshi, New York, NY (US)

(72) Inventor: Vikram Joshi, New York, NY (US)

(73) Assignee: Vikram Joshi, New York, NY (US)

(21) Appl. No.: 19/433,028

(22) Filed: Dec. 25, 2025

Related U.S. Application Data
(60) Provisional application No. 63/818,876, filed on Jun. 6, 2025.

(51) Int. Cl. A61B 5/16; A61B 5/00; A61B 5/0533

(52) U.S. Cl. CPC A61B 5/165; A61B 5/0077; A61B 5/0533; A61B 5/4803; A61B 2503/12

(57) ABSTRACT

A system and method for objectively determining interpersonal compatibility by correlating synchronized multimodal biometric responses to humorous stimuli. The system presents categorized media content to a user while simultaneously capturing asynchronous data streams, including facial video, audio vocalizations, and galvanic skin response (GSR). A synchronization engine aligns these streams, applying a latency correction algorithm to correlate delayed physiological arousal peaks (phasic SCR) with antecedent visual micro-expressions (Action Units 6 and 12). By validating visual smiles with physiological arousal, the system distinguishes genuine Duchenne responses from social masking. These validated responses are fused via a tensor network into a multidimensional “Humor Response Vector.” Compatibility is determined by calculating the cosine similarity between users' vectors in a latent space, providing a quantification of psychological alignment that transcends self-reported bias. Embodiments include privacy-preserving edge extraction and homomorphic encryption of profile vectors.

### CROSS-REFERENCE TO RELATED APPLICATIONS

This application claims the benefit of priority to U.S. Provisional Patent Application No. 63/818,876, filed Jun. 6, 2025, entitled “System and Method for Humor-Based Compatibility Matching,” the entire contents of which are hereby incorporated by reference.

### FIELD OF THE INVENTION

The present disclosure relates generally to the field of affective computing and signal processing. More specifically, the invention relates to systems and methods for synchronizing asynchronous biometric data streams-including computer vision-based facial action unit detection, electrodermal activity, and spectral audio analysis—to generate high-dimensional psychometric vector embeddings for the objective quantification of interpersonal compatibility.

### BACKGROUND OF THE INVENTION

### 1. Limitations of Conventional Compatibility Systems

The field of automated matchmaking has historically relied on explicit user inputs. Conventional systems typically employ questionnaires wherein users self-report their preferences (e.g., “I enjoy sarcasm”) or static profile attributes (e.g., age, location, interest tags). These systems suffer from several fundamental technical and epistemological flaws. First, self-reported data is inherently subjective and prone to “social desirability bias,” where users select answers, they believe are attractive rather than accurate. Second, static profiles fail to capture the dynamic, temporal nature of human emotional response. A user's claim that they enjoy “dark humor” is a low-fidelity data point compared to the observable physiological reality of their reaction to a specific dark humor stimulus.

Furthermore, existing “swipe-based” mechanics focus on user interface (UI) interactions rather than the underlying psychological signal. While these systems effectively organize user activity, they do not solve the technical problem of verifying the authenticity of a user's reported preference. The industry lacks a mechanism for quantifying deep psychological compatibility through passive, objective signal acquisition.

### 2. The Technical Challenge of Affective Computing

Affective computing aims to enable processors to recognize and interpret human affect. However, accurate emotion detection faces the “Multimodal Asynchronicity Problem.” A genuine humor response involves a complex cascade of biological events: a cognitive resolution of incongruity (brain), a motor response (facial muscle contraction), a respiratory response (laughter), and an autonomic nervous system response (arousal/sweating).

These events do not occur simultaneously. Facial micro-expressions can manifest within 200 milliseconds of a stimulus. In contrast, the Galvanic Skin Response (GSR), driven by the sympathetic nervous system's activation of eccrine sweat glands, exhibits a latency of 1 to 3 seconds (the “sudomotor nerve delay”). Conventional systems that simply timestamp data streams without algorithmic lag correction fail to correctly correlate the visual smile with the physiological arousal, leading to false positives (e.g., interpreting a “polite smile” as genuine enjoyment). There is, therefore, a need for a system that not only captures these multimodal signals but also technically synchronizes and fuses them to distinguish between “Duchenne” (genuine) and non-Duchenne (social) humor responses, creating a verifiable vector of compatibility.

While multimodal sentiment analysis and automated matchmaking are known in the art, existing systems fail to account for the physiological asynchronicity between different biological streams. Specifically, prior art lacks a mechanism to calibrate for the 1.0 to 3.0 second sudomotor latency delay inherent in GSR validation. This results in high false-positive rates because such systems attempt to align visual and autonomic data using concurrent timestamps, failing to verify genuine emotional responses.

### BRIEF SUMMARY OF THE INVENTION

Conventional synchronization methods relying on simple timestamp alignment fail to account for this biological lag. The present invention solves this technical problem by enforcing a specific ‘Sudomotor Latency Window’ (1000-3000 ms) as a validation gate. This ensures that the system correlates a visual smile at time t only with the specific physiological arousal event at time t+Δ, thereby filtering out ‘social masking’ artifacts that lack the requisite autonomic delay.

The present invention addresses the aforementioned limitations by providing a system and method for generating a “Humor Response Vector” (HRV) through the synchronized fusion of asynchronous biometric data. Unlike prior art that relies on declarative data (what users say), the present invention relies on procedural data (how users react).

In one aspect, the system comprises a Humor Stimulus Engine that serves a sequence of media items tagged according to a computational humor taxonomy (e.g., the Humor Styles Questionnaire dimensions: Affiliative, Self-Enhancing, Aggressive, Self-Defeating).

In a second aspect, the system comprises a Multimodal Capture Interface that simultaneously records:
(i) Visual Data: Using a Convolutional Neural Network (CNN) or facial mesh topology to detect specific Facial Action Coding System (FACS) Action Units, specifically identifying the concurrent activation of AU6 (Orbicularis Oculi) and AU12 (Zygomaticus Major)—the physiological signature of a Duchenne smile.
(ii) Physiological Data: Utilizing a wearable sensor to measure Electro-Dermal Activity (EDA). The system applies a convex optimization algorithm to decompose the raw EDA signal into a slow-moving Tonic component (Skin Conductance Level—SCL) and a fast-moving Phasic component (Skin Conductance Response—SCR), isolating the specific arousal peaks associated with humor stimuli.
(iii) Audio Data: Analyzing spectral flux and Mel-Frequency Cepstral Coefficients (MFCCs) to detect the harmonic “burst” signatures of laughter.

In a third aspect, the system includes a Synchronization and Fusion Engine. This engine applies a sliding-window correlation algorithm to align the delayed physiological peaks with the antecedent visual triggers. It then utilizes a Tensor Fusion Network to mathematically combine the visual, audio, and physiological vectors into a unified representation. This fusion allows the system to differentiate complex emotional states—for example, distinguishing “nervous laughter” (High Audio+High Arousal+Fear Micro-expression) from “joyful laughter” (High Audio+High Arousal+Duchenne Smile).

Finally, the system generates a compatibility score by computing the Cosine Similarity between the Humor Response Vectors of two users in a high-dimensional latent space. This technical approach transforms the abstract idea of “compatibility” into a measurable, calculable geometric relationship between objective data structures.

A key technical advantage of the present invention is the implementation of a Sudomotor Latency Window that acts as a temporal verification gate. This specific 1000-3000 ms window-confirmed by established biological data—is not a mere design choice but a technical requirement to correctly fuse motor-based facial cues with autonomic responses, thereby distinguishing genuine Duchenne humor from social masking.

### BRIEF DESCRIPTION OF THE DRAWINGS

FIG. 1 is a block diagram illustrating the networked system architecture, detailing the division of labor between the Server System 104 and the Client Device 102 connected via a Network 106. The diagram further details the Sensor Array 108 for raw capture and the Matchmaking Engine 116 residing on the server.

FIG. 2 is a timing diagram of the Multimodal Synchronization Pipeline, illustrating the temporal offset between the Smile Onset 202 (video) and the Phasic Peak 206 (GSR) across the Sudomotor Latency Window 204.

FIG. 3 is a flow diagram illustrating the logic for validating humor responses, proceeding from multimodal capture 304 to Duchenne classification 318 or masking classification 316.

FIG. 4 illustrates the Signal Decomposition Algorithm, showing the separation of the Raw EDA Signal 402 into a drifting Tonic Component 404 and an event-related Phasic Component 406.

FIG. 5 is a conceptual diagram of the Tensor Fusion Network, depicting the outer product operation 508 applied to unimodal vectors 502, 504, 506 to generate the Multimodal Affect Tensor 510.

FIG. 6 depicts the Vector Space Model, illustrating the calculation of cosine similarity θ between a User A Vector 602 and a User B Vector 604 within the humor taxonomy space.

FIG. 7 is a data flow diagram of the Privacy-Preserving Architecture, detailing the trusted edge environment 700 where Raw Biometric Capture 702 occurs. The figure illustrates the transition from cleartext Vector v to Homomorphic Encryption 708, passing Encrypted Ciphertext to the untrusted cloud server 750 for processing by the Blind Matchmaking Module 752.

### DETAILED DESCRIPTION OF THE PREFERRED EMBODIMENTS

### 1. System Architecture and Definitions

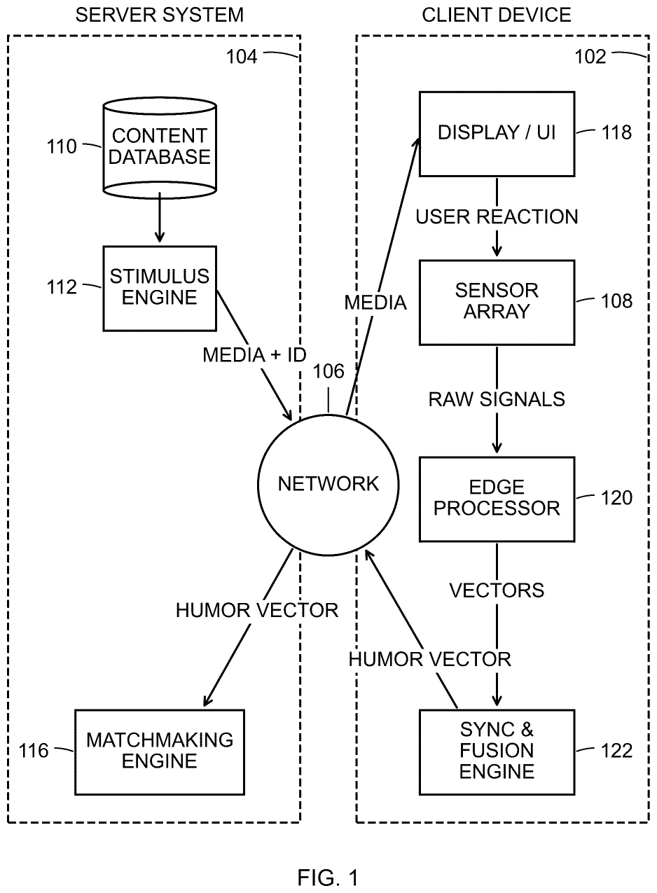

Referring to FIG. 1, the system 100 comprises a Client Device 102 and a Server System 104 connected via a Network 106. The Server System 104 includes a Content Database 110 storing tagged media and a Stimulus Engine 112 responsible for selecting and serving content sequences (Media+ID) to the client.

The Client Device 102, which may be a smartphone, AR headset, or desktop computer, is equipped with a Display/UI 118 for presenting stimuli and a Sensor Array 108 for capturing user reactions. The Sensor Array 108 captures raw signals (video, audio, EDA) which are passed to an Edge Processor 120. Crucially, the Edge Processor 120 performs feature extraction locally, converting raw biometric data into feature vectors. These vectors are passed to the Sync & Fusion Engine 122, which aligns the asynchronous signals and generates the final Humor Vector. This Humor Vector is transmitted via Network 106 to the Matchmaking Engine 116 residing on the Server System 104. This architecture ensures that high-bandwidth, privacy-sensitive raw data never leaves the Client Device 102.

### 2. The Humor Stimulus Module

The Server System 104 maintains a Content Database 110 containing media elements (video clips, memes, audio snippets). Each element is tagged with a vector representation corresponding to a humor taxonomy. In the preferred embodiment, the system utilizes the Humor Styles Questionnaire (HSQ) taxonomy developed by Martin et al., which categorizes humor into four dimensions:
(i) Affiliative: Humor used to enhance social cohesion (e.g., harmless jokes).
(ii) Self-Enhancing: Humor used to maintain a positive outlook on life (e.g., finding the bright side).
(iii) Aggressive: Humor used to tease or disparage others (e.g., sarcasm, satire).
(iv) Self-Defeating: Humor used to ingratiate oneself at one's own expense.

To facilitate vector operations, the system maps specific content metadata to these dimensions using a weighted scheme. For instance, content tagged as “Puns” or “Wordplay” is mapped primarily to the Affiliative dimension with a secondary weight in the Self-Enhancing dimension. Content tagged as “Roast” or “Satire” maps primarily to the Aggressive dimension. Complex categories such as “Dark” or “Morbid” humor are mapped as a composite vector summing components of the Aggressive dimension and the Self-Enhancing dimension (representing a coping mechanism). Conversely, “Wholesome” content positively reinforces the Affiliative dimension while applying a negative weight to the Aggressive dimension.

The Content Database 110 serves this content to the Client Device 102 via a Contextual Bandit Algorithm (e.g., Thompson Sampling), which optimizes the sequence of stimuli to maximize information gain regarding the user's specific humor vector.

### 3. Multimodal Biometric Capture

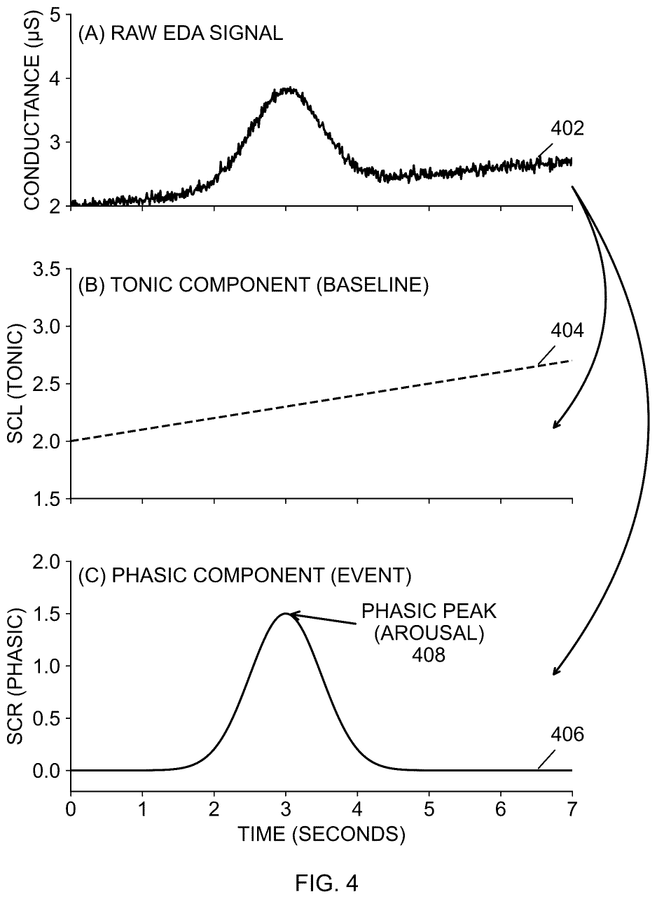

Upon presentation of a stimulus, the Sensor Array 108 initiates data capture.
(i) Visual Capture: The front-facing camera captures video frames at 30-60 fps. An on-device Facial Landmark Detector (e.g., Google MediaPipe Face Mesh) extracts 468 3D landmarks. The system calculates the Euclidean distance between specific landmarks to determine the intensity of FACS Action Units. Specifically, the “Eye Aspect Ratio” (EAR) and “Mouth Aspect Ratio” (MAR) are computed to quantify the contraction of AU6 and AU12.
(ii) Physiological Capture: A wearable peripheral (e.g., a smartwatch or ring) measures Galvanic Skin Response (GSR). The raw signal is sampled at a frequency of roughly 4-10 Hz. As shown in FIG. 4, a signal processing module applies a low-pass filter (e.g., a Butterworth filter) to remove motion artifacts and 50/60 Hz power line noise.
(iii) Audio Capture: The microphone captures vocalizations. The audio stream is segmented into frames (e.g., 25 ms windows). The system extracts features including Zero-Crossing Rate (ZCR), Root Mean Square (RMS) energy, and Mel-Frequency Cepstral Coefficients (MFCCs). A binary classifier (e.g., Support Vector Machine or LSTM) determines the probability of the frame containing laughter versus speech or silence.

### 4. Synchronization and Signal Decomposition

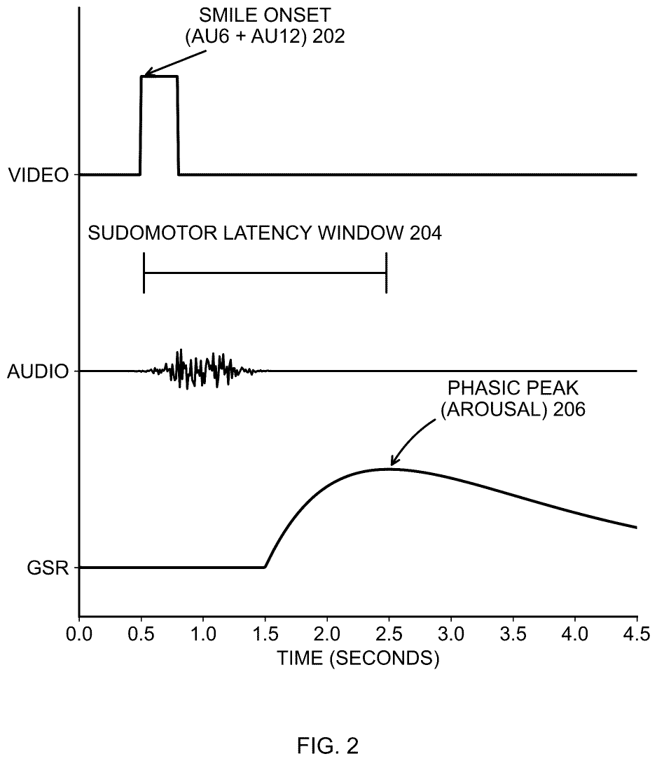

A critical innovation is the Synchronization Engine illustrated in FIG. 2, which addresses the “Multimodal Asynchronicity Problem.” Biometric streams occur on different biological timelines. The Smile Onset 202 (visual trigger) establishes a reference timestamp t_{ref}, typically manifesting with an onset latency of 200-500 ms post-stimulus. Audio vocalizations (laughter) typically follow at approximately 400-800 ms relative to the stimulus.

Unlike prior art systems that align multimodal signals based on concurrent timestamps, the system applies a rigid Sudomotor Latency Window 204 to correlate the visual data with the corresponding autonomic response. This window is not arbitrary but is technically calibrated to the sudomotor nerve delay, rejecting any physiological peaks that occur outside the 1000-3000 ms offset as noise or unrelated events. Specifically, the Galvanic Skin Response (GSR) exhibits an onset latency of 1000-3000 ms, with the peak amplitude occurring between 2000-5000 ms. If a heart rate (PPG) sensor is utilized for secondary validation, the system accounts for a latency of 2000-5000 ms.

To isolate the relevant physiological data, the system employs a signal decomposition algorithm as illustrated in FIG. 4. The Raw EDA Signal 402 often contains baseline drift caused by environmental factors or metabolic changes. The system decomposes this raw signal into two distinct components: a Tonic Component 404 (representing the slowly changing Skin Conductance Level or baseline) and a Phasic Component 406 (representing the rapid Skin Conductance Response). By subtracting the Tonic Component 404, the system isolates the Phasic Peak 408. It is noted that this isolated Phasic Peak 408 (shown in the signal decomposition of FIG. 4) corresponds structurally and temporally to the Phasic Peak 206 referenced in the timing diagram of FIG. 2. The system searches for this peak specifically within the look-ahead window (t_{ref}+Δ) defined by the latency parameters described above. If the peak magnitude exceeds a threshold τₐᵣₒᵤₛₐₗ, the system validates the preceding visual smile as a genuine “High Arousal” response; otherwise, it is flagged as “Low Arousal” or social masking.

The technical necessity of the specific latency correction utilized by the Synchronization Engine is supported by established physiological reference data. Specifically: (i) Facial Micro-expressions exhibit an onset latency of less than 200 ms and a peak latency of 300-500 ms; (ii) Vocalizations such as laughter typically manifest at 400-800 ms; and (iii) Sudomotor activity (GSR) exhibits a significantly delayed onset latency of 1000-3000 ms with peak amplitude at 2000-5000 ms.

As demonstrated by this data, there is a clear biological lag where sudomotor activity is delayed by several seconds relative to the primary visual trigger. By hard-coding this 1.0 to 3.0 second ‘Sudomotor Latency Window’ into the Synchronization Engine, the system achieves a level of verification fidelity that simple concurrent timestamp-based alignment cannot attain.

### 5. Duchenne Detection and Validation Logic

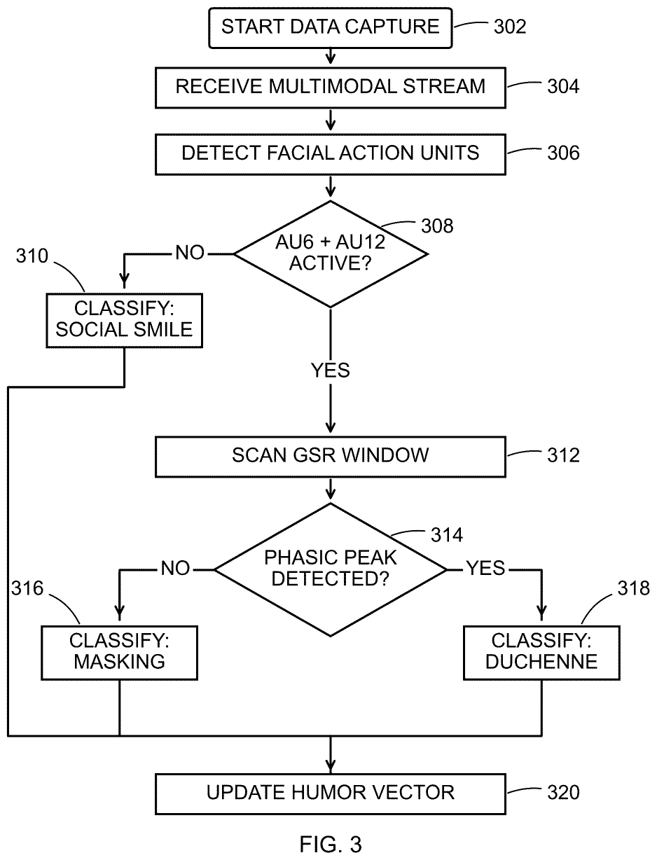

FIG. 3 illustrates the specific logic flow executed by the Edge Processor 120 to distinguish genuine humor from social politeness. The process begins at step 302 with the initiation of data capture. At step 304, the system receives the synchronized multimodal stream.

The system first executes facial analysis at step 306 to detect Facial Action Units. At decision block 308, the system determines if the “Duchenne Marker” is present, defined as the simultaneous activation of AU6 (Orbicularis Oculi) and AU12 (Zygomaticus Major). If these are not active, the system classifies the expression as a “Social Smile” at step 310 and assigns it a low or zero weight.

If the visual marker is present (“YES” at 308), the system proceeds to step 312 to scan the physiological data within the aforementioned latency window. At decision block 314, the system checks for the presence of a significant Phasic Peak (see 408 in FIG. 4) that exceeds a pre-determined arousal threshold.
(i) NO at 314: If no arousal peak is detected, the system classifies the response as “Masking” at step 316 (a visual smile lacking physiological emotion).
(ii) YES at 314: If the peak is confirmed, the system classifies the response as a genuine “Duchenne” response at step 318.

Finally, at step 320, the validated response data is forwarded to the fusion engine to update the user's humor vector.

### 6. Tensor Fusion and Vector Embedding

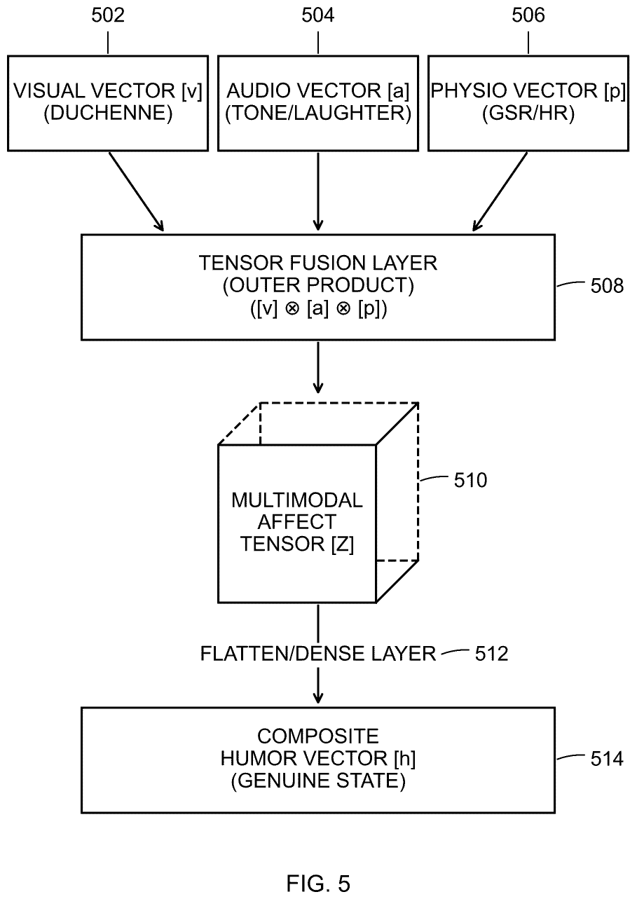

To capture the complex interplay between modalities, the system employs a Tensor Fusion Network as depicted in FIG. 5. The system inputs three unimodal feature vectors:
(i) Visual Vector [v] 502: Representing the intensity of the validated Duchenne expression.
(ii) Audio Vector [a] 504: Representing acoustic features of laughter (e.g., spectral flux, zero-crossing rate).
(iii) Physio Vector [p] 506: Representing the normalized amplitude of the Phasic Peak.

Rather than simply concatenating these vectors, the Tensor Fusion Layer 508 computes the tensor outer product (Z=[v]⊗[a]⊗[p]). This operation results in a Multimodal Affect Tensor [Z] 510 that explicitly models bimodal (e.g., audio-visual) and trimodal interactions. This tensor is then passed through a Flatten/Dense Layer 512 (a fully connected neural network layer) to project the high-dimensional correlations into a compact Composite Humor Vector [h] 514. This vector serves as the mathematical representation of the user's genuine emotional reaction to the specific stimulus.

### 7. The Matchmaking (Correlation) Engine

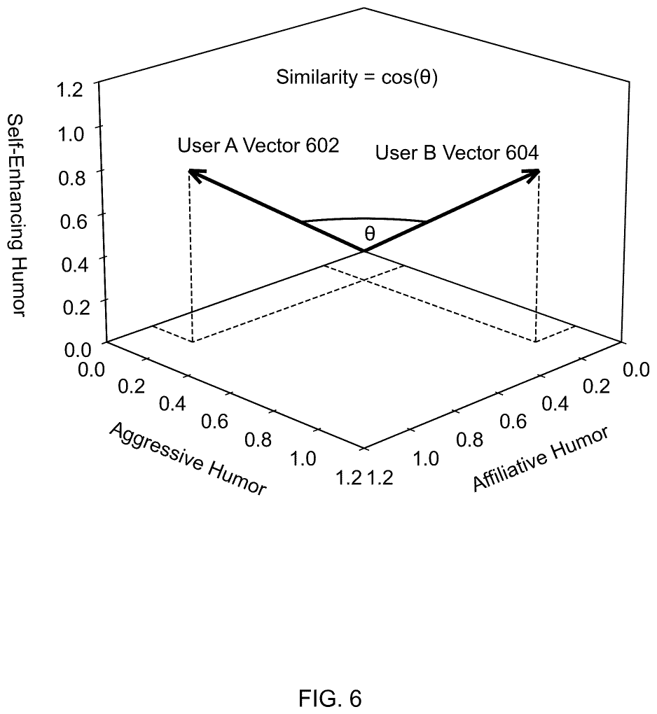

The matchmaking engine determines compatibility by analyzing the geometric relationship between user vectors in a high-dimensional space, as illustrated in FIG. 6.

Each user is represented by a coordinate in the humor taxonomy space (e.g., dimensions of Affiliative, Aggressive, Self-Enhancing, and Self-Defeating humor). FIG. 6 depicts a simplified 3D view where User A is represented by Vector 602 and User B is represented by Vector 604. The engine calculates the Cosine Similarity between these two vectors, represented by the angle θ. The similarity score is derived using the equation:

$$Similarity = cos ( θ ) = \frac{A \cdot B}{‖ A ‖ ‖ B ‖}$$

A smaller angle θ results in a cosine value closer to 1.0, indicating a high degree of alignment in humor appreciation and psychological compatibility.

### 8. Privacy and Encryption Architecture

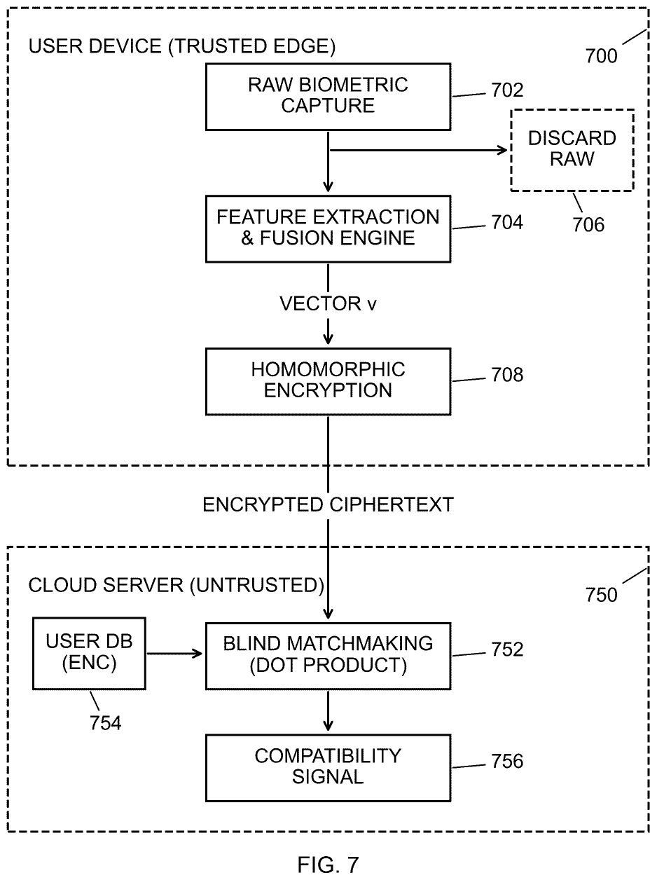

FIG. 7 details the privacy-preserving data flow. The architecture is strictly divided into a Trusted Edge Environment (User Device 700) and an Untrusted Cloud Server 750.

The process begins with Raw Biometric Capture 702. This raw data is processed immediately by the local Feature Extraction & Fusion Engine 704. Crucially, as indicated at step 706, the raw images and audio are discarded immediately after feature extraction and are never transmitted.

The resulting feature vector is then subjected to Homomorphic Encryption 708. This outputs an encrypted ciphertext which is transmitted to the Cloud Server 750 and stored in an Encrypted User Database 754. The Blind Matchmaking Module 752 performs vector operations (e.g., dot products) directly on this ciphertext. Specifically, the system utilizes a leveled Fully Homomorphic Encryption (FHE) scheme (such as CKKS) that supports approximate arithmetic operations on real numbers. The Blind Matchmaking Module 752 computes the scalar product of the encrypted user vector and the encrypted target vector by utilizing the homomorphic multiplication and addition properties of the scheme. This yields an encrypted result which, when decrypted by the client (or a trusted key management service), reveals only the final scalar compatibility score 756, ensuring the high-dimensional behavioral vectors remain mathematically opaque to the cloud provider. The server calculates the Compatibility Signal 756 without ever decrypting the underlying behavioral profile, thereby preserving user anonymity.

1. A computer-implemented method for synchronizing asynchronous biometric data streams to validate emotional authenticity in a computing system, the method comprising:
presenting, on a display device of a first user, a sequence of humor-based media stimuli, wherein each stimulus is associated with a specific humor taxonomy vector;
capturing, via a sensor array coupled to the first user, a plurality of asynchronous biometric data streams including a video stream of a facial region, an audio stream of vocalizations, and a physiological stream of electrodermal activity;
extracting a visual feature vector from the video stream, an audio feature vector from the audio stream, and a physiological feature vector from the physiological stream;
synchronizing, by a processor, the asynchronous biometric data streams to a common stimulus timeline by applying a latency-correction algorithm to align a phasic peak in the physiological stream with a visual event in the video stream;
detecting, in the synchronized video stream, a simultaneous activation of Facial Action Unit 6 (Orbicularis Oculi) and Facial Action Unit 12 (Zygomaticus Major) indicative of a Duchenne smile;
validating the Duchenne smile by determining if the aligned phasic peak in the physiological stream exceeds a predetermined arousal threshold calculated relative to a baseline tonic component, thereby generating a verified humor response;
fusing the verified humor response with the audio feature vector by computing a tensor outer product of the visual, audio, and physiological feature vectors to generate a multimodal tensor representation that captures inter-modal dependencies;
updating a first multidimensional humor profile vector associated with the first user based on the multimodal tensor representation;
calculating a vector similarity metric between the first multidimensional humor profile vector and a second multidimensional humor profile vector associated with a second user; and
generating a compatibility signal if the vector similarity metric exceeds a predetermined match threshold.

2. The method of claim 1, wherein the step of synchronizing comprises:
decomposing a raw signal from the physiological stream into a tonic skin conductance level (SCL) and a phasic skin conductance response (SCR);
identifying a timestamp of the phasic SCR peak; and
mapping the timestamp of the phasic SCR peak to a preceding timestamp in the video stream based on a predetermined sudomotor latency window of 1 to 4 seconds.

3. The method of claim 1, wherein fusing the verified humor response comprises:
generating a visual feature vector, an audio feature vector, and a physiological feature vector; and
computing a tensor outer product of the visual, audio, and physiological feature vectors to generate a multimodal tensor representation that captures inter-modal dependencies.

4. The method of claim 1, wherein the humor taxonomy vector corresponds to dimensions selected from the group consisting of affiliative humor, self-enhancing humor, aggressive humor, and self-defeating humor.

5. The method of claim 1, further comprising:
encrypting the first multidimensional humor profile vector using a fully homomorphic encryption scheme prior to calculating the vector similarity metric; and
performing the calculation of the vector similarity metric in an encrypted domain such that the first multidimensional humor profile vector remains unexposed to a server performing the calculation.

6. The method of claim 1, wherein the detecting of the simultaneous activation utilizes a convolutional neural network (CNN) resident on a processor coupled to the display device to extract facial landmarks in real-time, and wherein raw image data is discarded subsequent to landmark extraction to preserve user privacy.

7. A system for objective compatibility determination, comprising:
a client device comprising a display, a camera, and a galvanic skin response (GSR) sensor;
an edge processor operational on the client device and configured to:
receive a video stream from the camera and a GSR signal from the GSR sensor;
detect a simultaneous activation of eye-orbit muscles and lip-corner muscles; and
decompose the GSR signal to isolate a phasic conductance component;
a synchronization and fusion engine operational on the client device and configured to:
validate the simultaneous activation by verifying the presence of a peak in the phasic conductance component within a sudomotor latency window; and
fuse the validated activation with the peak magnitude to generate a humor vector; and
a server system in network communication with the client device, comprising a content database storing a plurality of media items, wherein each media item is mapped to a pre-computed target vector; and a matchmaking engine configured to:
receive the humor vector; and
calculate a cosine similarity between the humor vector and the target vector corresponding to a presented media item to determine a compatibility score.

8. The system of claim 7, wherein the server system utilizes a reinforcement learning agent to select the sequence of content items, using the intensity of the validated contractions as a reward signal to optimize the exploration of the user's humor preferences.

9. A non-transitory computer-readable medium storing instructions that, when executed by a processor, cause the processor to perform operations comprising:
accessing a first vector embedding representing a first user's physiological responsiveness to a set of humor categories;
accessing a second vector embedding representing a second user's physiological responsiveness to multi-dimensional vector space defining the set of humor categories; and
computing a cosine similarity score between the first vector embedding and the second vector embedding, wherein the first and second vector embeddings are derived from a tensor fusion of time-aligned facial micro-expressions and galvanic skin response peaks captured during media consumption.
